(19) United States
(12) Patent Application Publication
(10) Pub. No.: US 2013/0118507 A1
(43) Pub. Date: May 16, 2013

(54) Bite guard and method of use

(71) Applicant: James L. Chappuis, Atlanta, GA (US)

(72) Inventor: James L. Chappuis, Atlanta, GA (US)

(21) Appl. No.: 13/676,883

(22) Filed: Nov. 14, 2012

Related U.S. Application Data
(60) Provisional application No. 61/559,420, filed on Nov. 14, 2011.

(51) Int. Cl. A61B 17/24

(52) U.S. Cl. CPC A61B 17/24
USPC 128/859

(57) ABSTRACT

A bite guard to protect an individual's teeth, tongue, and/or mouth during medical procedures and tests. The bite guard is designed to be placed in an individual's mouth between the upper and lower set of teeth during certain medical procedures such as motor evoked potential (MEP) testing which is known to cause an individual's jaws muscles to contract. The present invention will protect the teeth, tongue and mouth of an individual by preventing the upper and lower teeth from coming in contact while maintaining a bridged opening for the placement of tubular inserts as well as maintain unobstructed breathing space.

### REFERENCE TO RELATED APPLICATIONS

In accordance with 37 C.F.R. 1.76, a claim of priority is included in an Application Data Sheet filed concurrently herewith. Accordingly, under 35 U.S.C. §119(e), 120, 121, and/or 365(c) this present invention claims priority to U.S. Provisional Patent Application No. 61/559,420, entitled “Bite Guard and Method of Use”, filed Nov. 14, 2011. The contents of which the above referenced application is incorporated herein by reference.

### FIELD OF THE INVENTION

The present invention relates to bite guards and, in particular, to bite guards which are used to protect an individual's mouth during various surgical procedures.

### BACKGROUND OF THE INVENTION

An evoked potential or evoked response is an electrical potential recorded from an individual's nervous system upon the presentation of a stimulus, as opposed to a spontaneous potential which are detected by electroencephalography (EEG) or electromyography (EMG). Signals from an evoked potential can be recorded from cerebral cortex, brain stem, spinal cord, and peripheral nerves. These signals are recorded from the central nervous system following the stimulation of sense organs. For example, visual evoked potentials resulting from a flashing light of changing pattern on a display; auditory evoked potentials resulting from a click or tone stimulus presented through earphones. Additionally, there can be tactile or somatosensory evoked potential resulting from tactile or electrical stimulation of a sensory or mixed nerve in the periphery.

Motor evoked potentials (MEP) are recorded from muscles following a direct stimulation of exposed motor cortex, or trans cranial stimulation of motor cortex, either magnetic or electrical. Trans cranial magnetic MEP (TCmMEP) potentially offers clinical diagnostic applications. Trans cranial electrical MEP (TCeMEP) has been in widespread use for several years for intraoperative monitoring of pyramidal tract functional integrity.

Because MEP amplitude is correlated with motor excitability, they offer a way to quantitatively test the role of various types of intervention on the motor system (pharmacological, behavioral, lesion, etc.) TMS-induced MEPs may thus serve as an index of covert motor preparation or facilitation, e.g. induced by the mirror neutron system when seeing someone else's actions. In addition, MEPs are used as a reference to adjust the intensity of stimulation that needed to be delivered by TMS when targeting cortical regions whose response might not be as measurable, e.g. in the context of TMS-based therapy.

Advances in MET are illustrated in U.S. Pat. No. 7,104,947 to Riehl which discloses a device wherein induced movement in a patient is detected and correlated with a TMS stimulating pulse so as to determine the patient's motor threshold stimulation level.

Should a catheter be inserted for use during the procedure, the patient can be at risk should a tightening of the jaw occur. Catheters are employed by medical personnel for many purposes such as providing for the passage of fluids, including gases, to and from the human body during such procedures. An endotracheal tube can be inserted through the oral cavity of a patient and into the trachea, for example, to provide for the supply of fluids to the body, for the monitoring of internal conditions in the body and for removal of secretions from within the body. Other examples of catheters include respiratory tubes for laryngeal masks, oral gastric tubes, and esophageal stethoscopes. A problem is that the catheter is usually relatively easy to deform as it passes between the patient's teeth when inserted orally. A problem during intubation is that a patient's teeth suffer dental trauma from being hit and/or jarred. This type of dental trauma results in claims against anesthesiologists so it is desirable to find a device capable of protecting a patient's teeth from dental trauma.

U.S. Published Patent Application No. 2010/0154791 discloses a medical device which includes a bite guard assembly configured to restrict sliding movement on an elongated member relative to the bite guard assembly. The bite guard assembly may include a main portion including a mouthpiece. The main portion may include a surface defining passageway for receipt of the elongated member there through. The bite guard assembly may also include a securing portion configured to restrict movement of the elongated member through the passageway and that is configured to be disposed between the surface and the elongated member.

U.S. Pat. No. 5,174,284 and U.S. Pat. No. 5,655,519 disclose a bite block for use with laryngeal mask airways, oral endotracheal tubes having upper non-incisor and lower non-incisor teeth engagement surfaces.

U.S. Pat. No. 6,890,322 discloses a device for securing a catheter, such as an endotracheal tube, with respect to a patient's mouth while protecting the catheter against occlusion by the patient's teeth.

Presently there exists a lack of consistent protection for the tongue and/or teeth of a patient during various surgical procedures, such as MEP. When applying such an electrical potential, contraction of the patient's muscles can cause the jaw muscles to contract, thereby potentially injuring the tongue and/or teeth of the patient as the jaw muscles are clenched.

### SUMMARY OF THE INVENTION

Disclosed is a device to protect an individual's mouth during medical procedures and tests. The bite guard is designed to be placed in an individual's mouth between the upper and lower sets of teeth during certain medical procedures such as motor evoked potential (MEP) testing. MEP testing can cause an individual's jaws muscles to contract and potentially injure the individual's tongue and/or teeth. The present invention will protect the teeth, tongue and mouth of an individual by preventing the upper and lower teeth from coming in contact with each other as a result of electrical stimulation from medical procedures. The device may also be used for any therapeutic or diagnostic procedure including the use of guide tubes, catheters or other medical devices including an endoscope that may be inserted into the body through the mouth.

Accordingly, it is an objective of the present invention to provide a bite guard to protect an individual's teeth, tongue, and/or mouth during various medical procedures.

It is a further objective of the present invention to provide a bite guard which can be used during certain medical procedures and can also permit the use of an endotracheal tube during these medical procedures.

It is yet another objective of the present invention to provide a bite guard which can be custom fit or customized to an individual's mouth and/or teeth so as to provide the best possible protection for the individual's teeth, tongue, and/or mouth during certain medical procedures.

It is a still further objective of the present invention to provide a bite guard which can be used during certain medical procedures, can also permit the use of an endotracheal tube during these medical procedures and can provide for adjustability of the endotracheal tube after the bite guard and endotracheal tube have been inserted into an individual's mouth.

Another objective of the invention is to protect a patient's teeth during orotracheal intubation.

Another objective of the invention is to prevent occlusion of a catheter and thus avoid, for example, hypoxia, hypercarbia and/or negative pressure pulmonary edema.

An advantage of the invention is that it efficiently and effectively secures a catheter in place and maintains that placement position.

A further advantage of the invention is that medical personnel may quickly and easily adjust the depth of a catheter traveling through a patient's oral cavity.

A further advantage of the invention is a simple design and can be easily disposed.

A yet further advantage of the invention is that once placed in the patient's mouth no part extends beyond the lips of the patient.

Other objectives and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 is a front perspective view of the present invention;

FIG. 2 is a front view of the present invention;

FIG. 3 is a front perspective view of the present invention including an endotracheal tube;

FIG. 4 is a perspective view of the underside of the filler material used with the present invention;

FIG. 5 is a front perspective view of the present invention including endotracheal tubes passing through U-shaped support member; and

FIG. 6 is a front perspective view of an alternate embodiment of the present invention including block shaped support members.

### DETAILED DESCRIPTION OF THE INVENTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

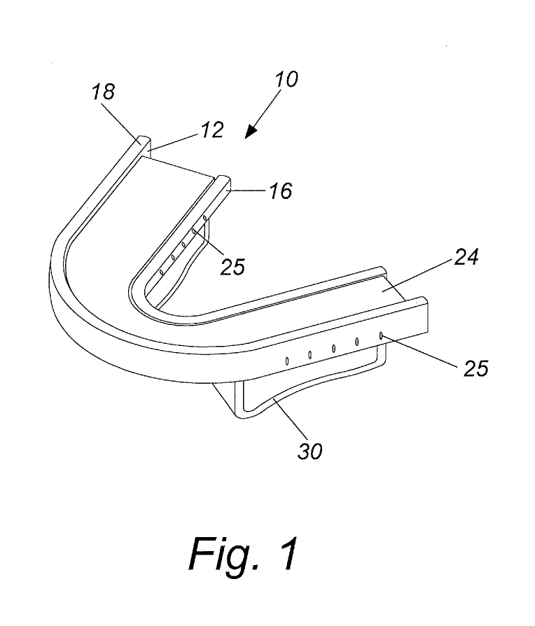

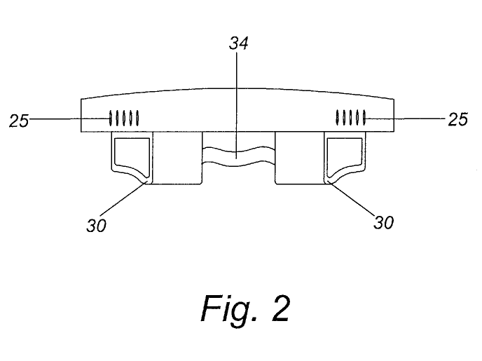

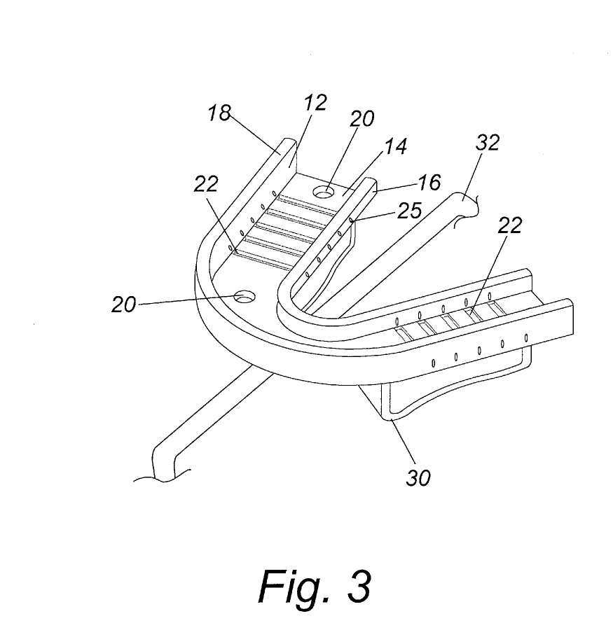

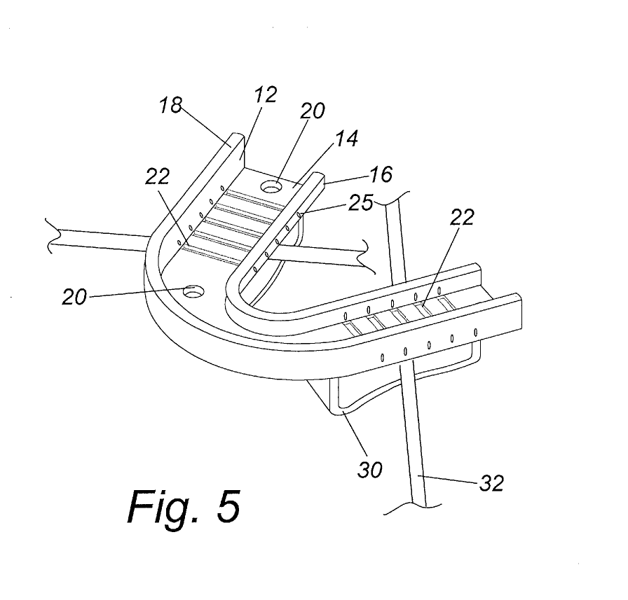

FIGS. 1-6, which are now referenced, illustrate the present invention and the manner in which it is used. The bite guard 10 of the present invention includes a channel 12. The channel is generally U-shaped so as to conform generally to the dental arc of an individual's teeth (not shown). The channel 12 includes a lower bottom 14 and two substantially side walls 16, 18 that extend at least partially along the inner and outer sides, respectively, of the bottom 14. In a preferred embodiment, the channel 12 includes a lower bottom 14 and two substantially side walls 16, 18 that extend completely along the inner and outer sides, respectively, of the bottom 14. In a preferred embodiment, the bottom 14 and side walls 16, 18 of the channel 12 are formed as an integral member. Alternatively, the bottom 14 and side walls 16, 18 could be initially formed as separated members and then secured together, as illustrated, to form channel 12. In a preferred embodiment, the lower bottom 14 is constructed of a material that is substantially resilient to absorb shock from the force of a jaw closure.

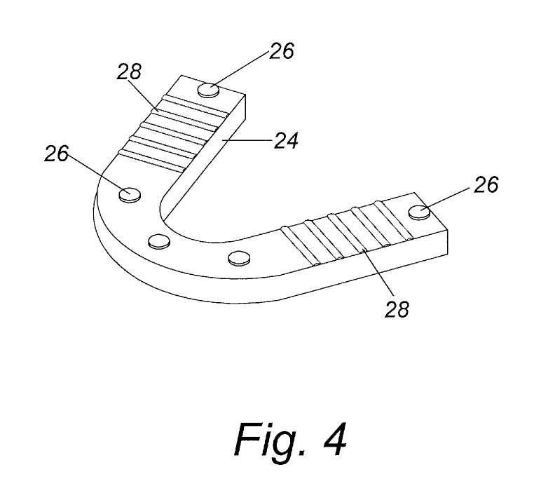

The bottom 14 of channel 12 includes a plurality of apertures 20 positioned along the longitudinal extent of the bottom 14. The bottom 14 further includes a plurality of troughs or depressions 22 positioned along the longitudinal extent of the bottom 14. The troughs 22 and apertures 20 are designed to interact with corresponding elements of a pliable, filler material 24. Filler material 24 is illustrated in FIGS. 1 and 4. Filler material 24 is preferable made from a pliable material such as rubber, soft plastic, silicone. In FIG. 4 the underside of the filler material 24 is illustrated. The underside of the filler material 24 includes substantially circular upstanding projections 26 and upstanding ridges 28. Projections 26 fit into apertures 20 and ridges 28 fit into troughs 22. The interaction between the projections 26, apertures 20, ridges 28 and troughs 22 assist in maintaining the filler material 24 in the channel 12 of the bite guard.

The filler material 24 enables an individual's upper teeth to fit comfortably into the bite guard and be securely held in the bite guard. The side walls 16, 18 of channel 12 include a plurality of apertures 25 therein. Apertures 25 also permit fluids, such as saliva, to drain away from the channel 12. The actual number of apertures 25 and placement thereof may vary and those depicted are for illustration only.

A U-shaped support or spacer 30 is formed by connecting 2 parallel leg pieces perpendicular to a joining base piece. In a preferred embodiment, the 2 leg pieces and the connecting base are formed as an integral U-shaped support or spacer 30.

At least one generally U-shaped support or spacer 30 is secured to an underneath portion of the bottom 14 of channel 12. In a preferred embodiment, the U-shaped support or spacer 30 is integrally formed with the bottom 14 of channel 12. In a preferred embodiment, a pair of generally U-shaped supports or spacers 30 are secured to an underneath portion of the bottom 14 of channel 12. In an alternative embodiment, a plurality of generally U-shaped supports or spacers 30 are secured to an underneath portion of the bottom 14 of channel 12. These supports or spacers 30 engage a row of an individual's teeth and prevent the bottom 14 of bite guard 10 from engaging the opposing row of teeth. The space between the upper and lower rows of teeth serves a plurality of purposes. First, the space permits the insertion of a tube into an individual's mouth. For instance, an endotracheal tube 32 may be inserted into an individual's mouth. Additionally, the opening between the bottom 14 of channel 12 and the base of generally U-shaped supports or spacers 30 is of an area sufficient to pass an endotracheal tube 32.

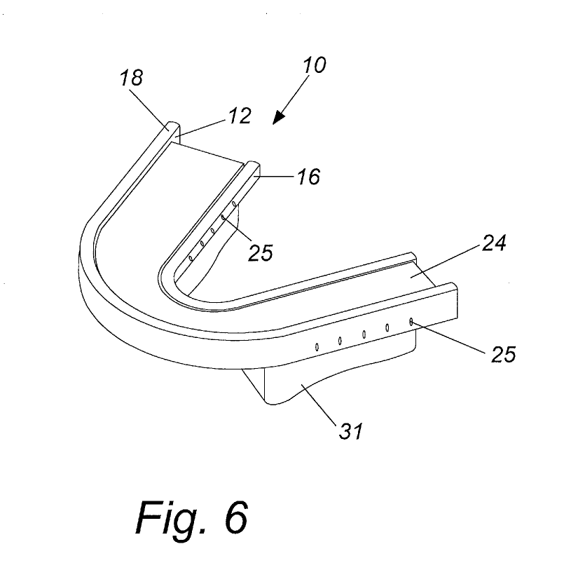

In another embodiment set forth in FIG. 6, at least one generally block shaped supports or spacers 31 are secured to an underneath portion of the bottom 14 of channel 12. In a preferred embodiment, the generally block shaped supports or spacers 31 are integrally formed with underneath portion of the bottom 14 of channel 12. In a preferred embodiment, a pair of generally block shaped supports or spacers 31 are secured to an underneath portion of the bottom 14 of channel 12. In an alternative embodiment, a plurality of generally block shaped supports or spacers 31 are secured to an underneath portion of the bottom 14 of channel 12. These supports or spacers 31 engage a row of an individual's teeth and prevent the bottom 14 of bite guard 10 from engaging the opposing row of teeth. The space between the upper and lower rows of teeth serves a plurality of purposes. First, the space permits the insertion of a tube into an individual's mouth. For instance, an endotracheal tube 32 may be inserted into an individual's mouth.

Another function of the supports or spacers 30 and 31 is to prevent an individual from biting their tongue in the event that an involuntary muscle movement is induced through a medical procedure, causing the upper and lower set of teeth to clench together.

In a preferred embodiment the supports or spacers 30 and 31 are substantially rigid to maintain their structural integrity under the force of an involuntary jaw closure. However, the supports or spacers 30 and 31 could also be resilient. Resilient supports would provide a more comfortable fit and feel in an individual's mouth. Additionally, resilient supports or spacers 30 and 31 can absorb shock, in the event that the upper and lower rows of an individual's teeth are quickly and violently brought together, as a result of a medical procedure or shock.

The channel 12 of the bite guard 10 can be made of a material which can be shaped or formed to fit an individual's mouth, similar to the mouth guard used by athletes. In this cast the bite guard is first custom fit to the individual's teeth and mouth and subsequently is utilized during medical procedures. This embodiment offers the individual greater protection and comfort because the bite guard is specifically designed or fit to their teeth and mouth. The material from which the channel 12 is made is available in various different colors, like red, orange, green, blue, etc. The colors can also be neon or the material of the bite guard can be a glow in the dark material. Further, the material from which channel 12 is made can be made to contain various flavors. The different colors and/or flavors would make the bite guard more acceptable to young children. For example, a physician could tell a young person that the bite guard was very tasty and delicious so the young person would not hesitate to use the bite guard during a medical procedure. Additionally, there could be indicia on the bite guard. For example the name of the manufacturer of the bite guard, the name of the hospital where the medical procedure is being performed, small friendly animals for young children, etc.

The bite guard may be made of any suitable material, preferably a biocompatible material such as plastic or rubber capable of being bitten without causing damage to the individual's teeth or gums.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A bite guard for medical procedures comprising:
a U-shaped base member conformed substantially to the dental arc of an individual's teeth; said base member defined by a top surface, a bottom surface, an inner surface and an outer surface; and at least one spacer support member secured to said bottom surface; whereby said top surface of said base member and bottom surface of said spacer support member cooperate with an individual's upper and lower teeth to maintain a space there between, constructed and arranged to accept a surgical tube.

2. The bite guard according to claim 1, wherein said base member includes a depending outer wall connected to said outer surface of said base member; said outer wall extending at least partially about said U-shaped base member.

3. The bite guard according to claim 1, wherein said base member includes a depending inner wall connected to the said inner surface of said base member; said inner wall extending at least partially about U-shaped base member.

4. The bite guard according to claim 1, wherein said inner wall, said outer wall and said U-shaped base member are integrally formed to create a channel of substantial dimension to accommodate a row of teeth and a filler piece.

5. The bite guard according to claim 1, said outer wall is continuous and extends completely about said U-shaped base member.

6. The bite guard according to claim 1, said inner wall is continuous and extends completely about said U-shaped base member.

7. The bite guard according to claim 1, wherein said base member has a plurality of apertures formed in said top surface to correspond with said filler piece.

8. The bite guard according to claim 1, wherein said base member has a plurality of troughs formed in said top surface to correspond with said filler piece.

9. The bite guard according to claim 1, wherein said top surface and said bottom surface are constructed of a material that is substantially resilient to absorb shock from the force of jaw closure.

10. The bite guard according to claim 1, wherein said filler piece is further defined as having a top surface, a bottom surface, an inner surface and an outer surface of a dimension substantially similar to said channel; said filler piece is constructed of a pliable material; said filler piece having a plurality of upstanding projections that interlock with the apertures in the said top surface and said filler piece having a plurality of upstanding ridges that interlock with the troughs on said top surface.

11. The bite guard according to claim 1, wherein said U-shaped base and said spacer support member are integrally formed.

12. The bite guard according to claim 1, wherein said spacer support member is further defined by two leg pieces extending parallel which are attached perpendicular to a joining base piece to form a U-shaped spacer support member.

13. The bite guard according to claim 12, wherein said leg pieces and said base piece are integrally formed to create said U-shaped spacer support member.

14. The bite guard according to claim 12, wherein said U-shaped spacer support members are constructed of a material that is substantially rigid so as to maintain structural integrity under the force of a jaw closure; said U-shaped space member creating an opening between the bottom surface of the U-shaped base member and the inner surface of the U-shaped spacer support member.

15. The bite guard according to claim 14, wherein said opening is of an area sufficient to insert a surgical tube.

16. The bite guard according to claim 1, wherein said inner wall has a plurality of apertures for drainage of liquids.

17. The bite guard according to claim 1, wherein said outer wall has a plurality of apertures for drainage of liquids.

18. The bite guard according to claim 1, wherein said spacer support member is defined by a top surface, a bottom surface and a plurality of side surfaces to form a block spacer support member.

19. The bite guard according to claim 18, wherein said block spacer support member is constructed of a material that is substantially rigid so as to maintain structural integrity under the force of jaw closure.
